(19) United States
(12) Patent Application Publication
(10) Pub. No.: US 2022/0249285 A1
(43) Pub. Date: Aug. 11, 2022

(54) TREATMENT SYSTEMS WITH ADJUSTABLE FLOW SHUNTS AND SENSORS, AND ASSOCIATED DEVICES AND METHODS

(71) Applicant: Shifamed Holdings, LLC, Campbell, CA (US)

(72) Inventors: Robert Chang, Belmont, CA (US); Claudio Argento, Felton, CA (US); Alice Yang, Campbell, CA (US)

(21) Appl. No.: 17/625,410

(22) PCT Filed: Jul. 8, 2020
(86) PCT No.: PCT/US2020/041159

§ 371 Date: Jan. 7, 2022

Related U.S. Application Data
(60) Provisional application No. 62/871,275, filed on Jul. 8, 2019.

(51) Int. Cl. A61F 9/007

(52) U.S. Cl. CPC A61F 9/00781

(57) ABSTRACT

The present technology is directed to systems for treating medical conditions, such as glaucoma, and associated devices and methods. For example, in some embodiments the present technology includes an adjustable flow shunt and a sensor. When implanted in a patient, the adjustable flow shunt can be configured to fluidly couple a first body region with a second body region such that it directs the flow of fluid from the first body region to the second body region. When implanted in the patient, the sensor can measure a physiologic parameter, such as intraocular pressure. The adjustable flow shunt can be adjusted based on the measurements taken by the sensor.

### CROSS-REFERENCE TO RELATED APPLICATION(S)

The present application claims priority to U.S. Provisional Patent Application No. 62/871,275, filed Jul. 8, 2019, the disclosure of which is incorporated by reference herein in its entirety.

### TECHNICAL FIELD

The present technology relates to systems for treating medical conditions and, in particular, to systems including an adjustable flow shunt and a sensor.

### BACKGROUND

Glaucoma is a degenerative ocular condition involving damage to the optic nerve that can cause progressive and irreversible vision loss. Glaucoma is frequently associated with ocular hypertension, an increase in pressure within the eye, and may result from an increase in production of aqueous humor (“aqueous”) within the eye and/or a decrease in the rate of outflow of aqueous from within the eye into the blood stream. Aqueous is produced in the ciliary body at the boundary of the posterior and anterior chambers of the eye. It flows into the anterior chamber and eventually into the capillary bed in the sclera of the eye. Glaucoma is typically caused by a failure in mechanisms that transport aqueous out of the eye and into the blood stream.

### BRIEF DESCRIPTION OF THE DRAWINGS

Many aspects of the present technology can be better understood with reference to the following drawings. The components in the drawings are not necessarily drawn to scale. Instead, emphasis is placed on illustrating the principles of the present technology. Furthermore, components can be shown as transparent in certain views for clarity of illustration only and not to indicate that the component is necessarily transparent. Components may also be shown schematically.

FIG. 1A is a simplified front view of an eye with an implanted shunt configured in accordance with an embodiment of the present technology.

FIG. 1B is an isometric view of the eye and implanted shunt of FIG. 1A.

FIG. 2 is a schematic illustration of a treatment system configured in accordance with embodiments of the present technology.

FIG. 3A is a schematic illustration of an implantable sensor configured in accordance with embodiments of the present technology.

FIG. 3B is a schematic illustration of an external device for communicating with the implantable sensor shown in FIG. 3A and configured in accordance with embodiments of the present technology.

FIG. 4 a graph illustrating the effective permeability of a ferrite antenna configured in accordance with embodiments of the present technology.

FIG. 5 is a graph illustrating the power efficiency for an inductive power transfer system configured in accordance with embodiments of the present technology.

FIG. 6 is a perspective view of an adjustable flow shunt configured in accordance with embodiments of the present technology.

FIGS. 7A-7D are partially schematic illustrations showing the operation of an actuation assembly for use with an adjustable flow shunt and configured in accordance with embodiments of the present technology.

FIGS. 8A-8D illustrate another adjustable flow shunt configured in accordance with embodiments of the present technology.

### DETAILED DESCRIPTION

The present technology is directed to systems for treating a medical condition and associated devices and methods. For example, in some embodiments the present technology includes an adjustable flow shunt and a sensor. When implanted in a patient, the adjustable flow shunt can be configured to fluidly couple a first body region and a second body region such that the shunt drains fluid from the first body region to the second body region. When implanted in the patient, the sensor can measure a physiologic parameter, such as pressure in the first body region. The adjustable flow shunt can be adjusted based, at least in part, on measurements taken by the sensor. In some embodiments, for example, the adjustable flow shunt adjusts flow through the shunt if the measured parameter is outside of a predetermined range. In other embodiments, the adjustable flow shunt adjusts flow through the shunt after a predetermined period of time and/or once a threshold is reached. In some embodiments, the sensor and/or the shunt are operably coupled to an external device, which can in some embodiments receive transmissions from the sensor, provide power to the sensor, display measurements taken by the sensor, determine adjustments for the shunt, and/or direct the shunt to adjust flow therethrough.

In some embodiments, the present technology is directed to systems for treating glaucoma and associated devices and methods. For example, in some embodiments the present technology includes an adjustable flow glaucoma shunt and a sensor. When implanted in a patient, the adjustable flow shunt can be configured to fluidly couple an anterior chamber of an eye with a target drainage location such that it drains aqueous from the anterior chamber and to the target drainage location. When implanted in the eye, the sensor can measure a physiologic parameter of the eye, such as intraocular pressure. The adjustable flow shunt can be adjusted based, at least in part, on measurements taken by the sensor. For example, in some embodiments the adjustable flow shunt adjusts flow through the shunt if the measured parameter is outside of a predetermined range. In other embodiments, the adjustable flow shunt adjusts flow through the shunt after a predetermined period of time and/or once a threshold is reached. In some embodiments, the sensor and/or the shunt are operably coupled to an external device, which can in some embodiments receive transmissions from the sensor, provide power to the sensor, display measurements taken by the sensor, determine adjustments for the shunt, and/or direct the shunt to adjust flow therethrough.

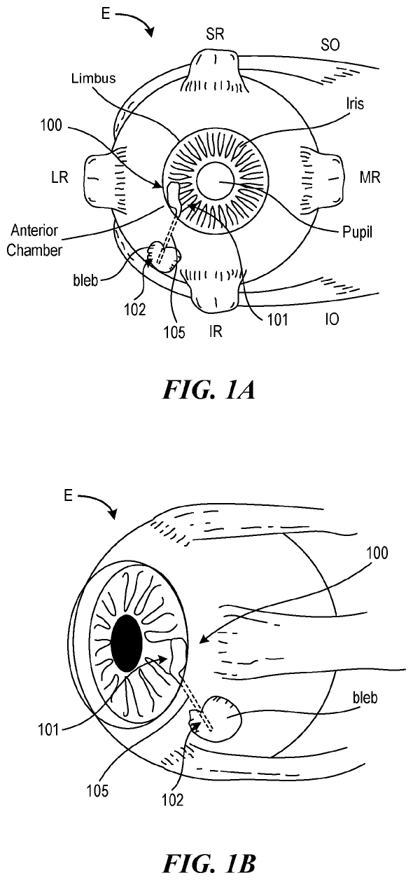

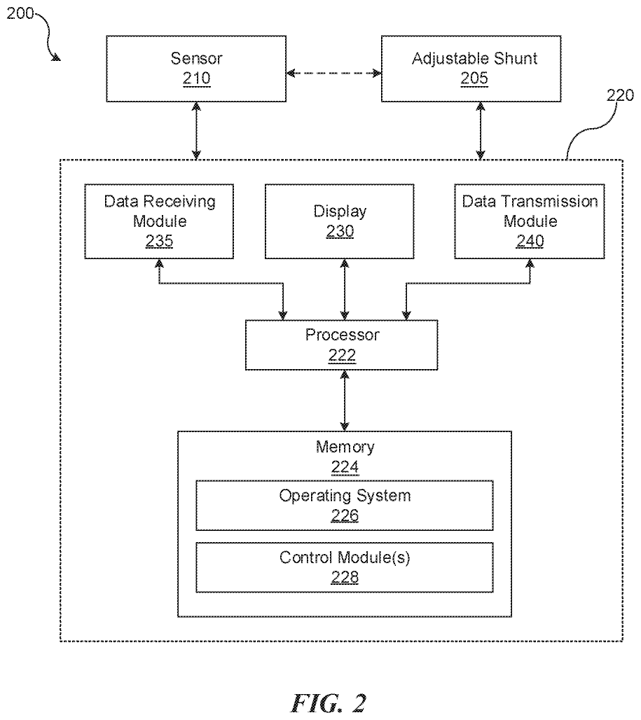

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to FIGS. 1A-8D.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%.

Although certain embodiments herein are described in terms of shunting fluid from an anterior chamber of an eye, one of skill in the art will appreciate that the present technology can be readily adapted to shunt fluid from and/or between other portions of the eye, or, more generally, from and/or between a first body region and a second body region. Moreover, while the certain embodiments herein are described in the context of glaucoma treatment, any of the embodiments herein, including those referred to as “glaucoma shunts” or “glaucoma devices” may nevertheless be used and/or modified to treat other diseases or conditions, including other diseases or conditions of the eye or other body regions. For example, the systems described herein can be used to treat diseases characterized by increased pressure and/or fluid build-up, including but not limited to heart failure (e.g., heart failure with preserved ejection fraction, heart failure with reduced ejection fraction, etc.), pulmonary failure, renal failure, hydrocephalus, and the like. Moreover, while generally described in terms of shunting aqueous, the systems described herein may be applied equally to shunting other fluid, such as blood or cerebrospinal fluid, between the first body region and the second body region.

The headings provided herein are for convenience only and do not interpret the scope or meaning of the claimed present technology.

### A. Intraocular Shunts for Glaucoma Treatment

Glaucoma refers to a group of eye diseases associated with damage to the optic nerve which eventually result in vision loss and blindness. As noted above, glaucoma is a degenerative ocular condition characterized by an increase in pressure within the eye resulting from an increase in production of aqueous within the eye and/or a decrease in the rate of outflow of aqueous from within the eye into the blood stream. The increased pressure leads to injury of the optic nerve over time. Unfortunately, patients often do not present with systems of increased intraocular pressure until the onset of glaucoma. As such, patients typically must be closely monitored once increased pressure is identified even if they are not symptomatic. The monitoring continues over the course of the disease so clinicians can intervene early to stem progression of the disease. Monitoring pressure requires patients to visit a clinic site on a regular basis which is expensive, time-consuming, and inconvenient. The early stages of glaucoma are typically treated with drugs (e.g., eye drops). When drug treatments no longer suffice, however, surgical approaches can be used. Surgical or minimally invasive approaches primarily attempt to increase the outflow of aqueous from the anterior chamber to the blood stream either by the creation of alternative fluid paths or the augmentation of the natural paths for aqueous outflow.

FIGS. 1A and 1B illustrate a human eye E and suitable location(s) in which a shunt may be implanted within the eye E in accordance with embodiments of the present technology. More specifically, FIG. 1A is a simplified front view of the eye E with an implanted shunt 100, and FIG. 1B is an isometric view of the eye E and shunt 100 of FIG. 1A. Referring first to FIG. 1A, the eye E includes a number of muscles to control its movement, including a superior rectus SR, inferior rectus IR, lateral rectus LR, medial rectus MR, superior oblique SO, and inferior oblique 10. The eye E also includes an iris, pupil, and limbus.

Referring to FIGS. 1A and 1B together, shunt 100 can have a drainage element 105 (e.g., a drainage tube) positioned such that an inflow portion 101 is positioned in an anterior chamber of the eye E, and an outflow portion 102 is positioned at a different location within the eye E, such as a bleb space. Depending upon the design of the device, the outflow portion 102 can be placed in a number of different suitable outflow locations (e.g., between the choroid and the sclera, between the conjunctiva and the sclera, etc.).

Outflow resistance can change over time for a variety of reasons, e.g., as the outflow location goes through its healing process after surgical implantation of a shunt (e.g., shunt 100) or further blockage in the drainage network from the anterior chamber through the trabecular meshwork, Schlemm's canal, the collector channels, and eventually into the vein and the body's circulatory system. Accordingly, a clinician may desire to modify the shunt after implantation to either increase or decrease the outflow resistance in response to such changes or for other clinical reasons. For example, in many procedures the shunt is modified at implantation to temporarily increase its outflow resistance. After a period of time deemed sufficient to allow for healing of the tissues and stabilization of the outflow resistance, the modification to the shunt is reversed, thereby decreasing the outflow resistance. In another example, the clinician may implant the shunt and after subsequent monitoring of intraocular pressure determine a modification of the drainage rate through the shunt is desired. Such modifications can be invasive, time-consuming, and/or expensive for patients. If such a procedure is not followed, however, there is a high likelihood of creating hypotony (excessively low eye pressure), which can result in further complications. In contrast, intraocular shunting systems configured in accordance with embodiments of the present technology allow the clinician to selectively adjust the flow of fluid through the shunt after implantation without additional invasive surgical procedures.

### B. Select Embodiments of Treatment Systems

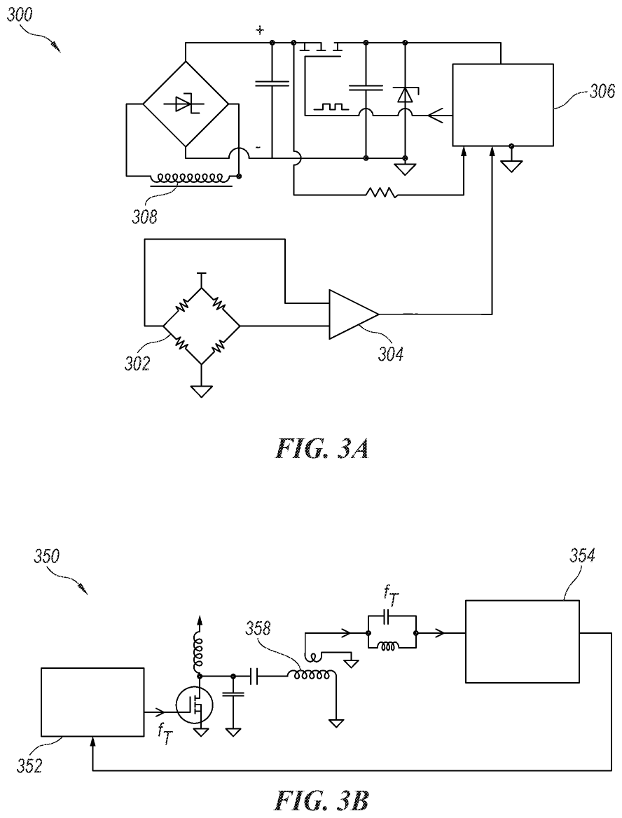

In some embodiments, the present technology provides systems for treating glaucoma or other medical conditions characterized by increased pressure and/or fluid collection. FIG. 2, for example, is a schematic diagram of a treatment system 200 (“system 200”) configured in accordance with embodiments of the present technology. The system 200 can include an adjustable shunt 205 and a sensor 210 configured to be implanted in a patient, and an external device 220 configured to remain external to the patient.

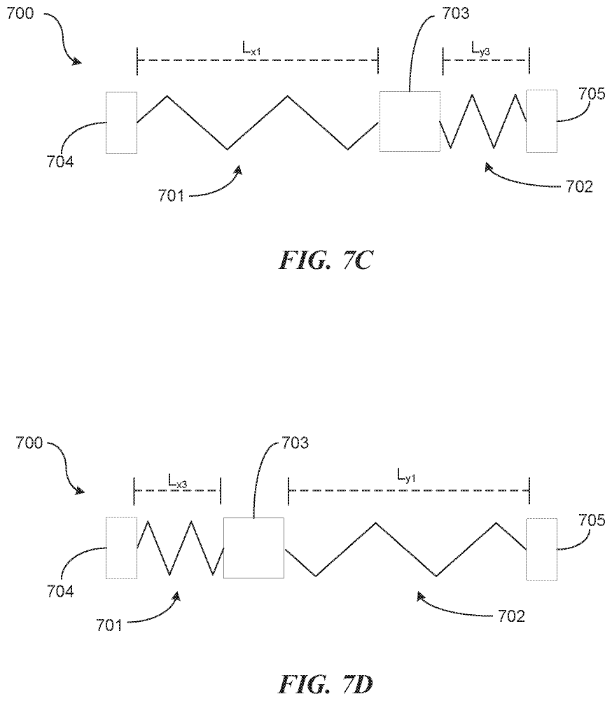

The adjustable shunt 205 can include an inflow region, an outflow region, and a flow control element configured to control fluid flow through the shunt between the inflow region and the outflow region. For example, when the adjustable shunt 205 is implanted in an eye of the patient, the inflow region can be positioned in fluid communication with an anterior chamber of the eye and the outflow region can be positioned in fluid communication with a target outflow/drainage location (e.g., a bleb space, a subconjunctival space, etc.). Accordingly, the adjustable shunt can route fluid (e.g., aqueous) from the anterior chamber of the eye to the target outflow location. The flow control element can move between at least a first position enabling a first amount of aqueous to flow through the shunt (and/or providing a first flow resistance through the shunt) and a second position enabling a second amount of aqueous to flow through the shunt (and/or providing a second flow resistance through the shunt) different than the first amount. Additional details of adjustable shunts configured in accordance with embodiments of the present technology are described with respect to FIGS. 6-8D.

The sensor 210 can be configured to be implanted in the eye to measure one or more physiological parameters of the patient. For example, the sensor 210 can be configured to measure an intraocular pressure in the anterior chamber or another location within the eye. The sensor 210 can also be configured to measure a rate of change of intraocular pressure in an anterior chamber or other location within the eye. The sensor 210 may also measure other suitable parameters that can be indicative of (e.g., correlated to) intraocular pressure. In some embodiments, the sensor 210 is physically coupled to (e.g., carried by, tethered to, etc.) the shunt 205. In other embodiments, the sensor 210 is spaced apart from and not physically coupled to the shunt 205. Regardless of whether the sensor is physically coupled to the shunt 205, the sensor 210 can be configured to communicate with the shunt 205 and/or the external device 220. The sensor 210 may communicate with the shunt 205 and/or the external device 220 via a wired or wireless connection (e.g., Bluetooth, WiFi, near-field-communication, frequency shift keying (“FSK”), on-of keying (“OOK”), etc.). In some embodiments, at least a portion of the sensor 210 is positionable within the anterior chamber of the eye of the patient. In some embodiments, the entire sensor 210 is positionable within the anterior chamber.

The sensor 210 can measure the one or more physiological parameters at various intervals. In some embodiments, for example, the sensor 210 measures the one or more physiological parameters at intermittent time intervals, such as once per minute, once per hour, twice per day, once per day, once per week, etc. In other embodiments, the sensor 210 continuously measures the one or more physiological parameters. In yet other embodiments, the sensor 210 provides “on-demand” measurements, in which the sensor 210 measures the one or more physiological parameters in response to a user (e.g., physician, nurse, patient, etc.) request to do so. In such embodiments, the sensor 210 can remain “off” until it is awakened and/or prompted to measure the one or more physiological parameters. The sensor 210 can also combine some or all of the foregoing operations, such as repeatedly measuring intraocular pressure once per day and also providing the capability to take “on-demand” measurements if requested to do so.

As provided above, the system 200 further includes the external device 220. In some embodiments, the external device can be a computing device, such as a smart phone, computer, tablet, or the like. The external device 220 can be configured to receive data from the sensor 210. For example, the external device 220 can include a data receiving module 235 for receiving measured physiological parameters from the sensor 210. The external device 220 can further include a display 230 (e.g., a monitor, touch screen, graphical user interface, etc.) that can display data received from the sensor 210. In some embodiments, the external device 220 can further include a data transmission module 240, which can send instructions to the adjustable shunt 205 (e.g., via wireless connection). In other embodiments, the data transmission module 240 is omitted, and the external device 220 does not directly communicate with the shunt 205. The external device can further include one or more processors 222 and memory 224 storing instructions executable by the one or more processors 222 to execute the functions described herein. For example, the memory 224 can include an operating system 226 and one or more control modules 228. The control modules 228 can store instructions that, when executed by the one or more processors, execute the various functions described herein.

The system 200 can be modified in a number of ways as will be apparent to one skilled in the art based on the disclosure herein. For example, although FIG. 2 illustrates a single external device 220, in alternative embodiments the external device 220 can instead be implemented as an external system encompassing a plurality of external devices, such that the operations described herein with respect to the external device 220 can instead be performed by the external system and/or the plurality of external devices. As another example, in some embodiments the system 200 may include additional components, such as a power transmitting module or element for charging the sensor 210. The power transmitting module can be included in the external device 220 or form a separate system component. As yet another example, in some embodiments the system 200 includes one or more intermediate devices that act as a relay hub between the sensor and the external device 220, and/or between the external device 220 and the shunt 205. Furthermore, in some embodiments, various components of the external device 220 can be omitted (e.g., the data transmission module 240).

In some embodiments, the system 200 provides a closed-loop operation for automatically adjusting flow through the shunt 205. For example, the sensor 210 can measure one or more physiological parameters in the eye at various time intervals (e.g., predetermined, on-demand, continuous, etc.) and transmit the measured values to the external device 220 (e.g., via the data receiving module 235). If the physiological parameter exceeds a first threshold, the external device 220 can direct the shunt 205 to adjust its flow control element to increase the amount of flow through the shunt 205. If the physiological parameter falls below a second threshold, the external device 220 can direct the shunt 205 to adjust its flow control element to decrease the amount of flow through the shunt 205. In some embodiments, the physiological parameter is an intraocular pressure, the first threshold corresponds to a maximum pressure, and the second threshold corresponds to a minimum pressure. In such embodiments, the first threshold can be between about 18 mmHg and 28 mmHg. For example, the first threshold can be about 18 mmHg, about 19 mmHg, about 20 mmHg, about 21 mmHg, about 22 mmHg, about 23 mmHg, about 24 mmHg, about 25 mm Hg, about 26 mmHg, about 27 mmHg, or about 28 mmHg. The second threshold can be between about 5 mmHg and 12 mmHg. For example, the second threshold can be about 5 mmHg, about 6 mmHg, about 7 mmHg, about 8 mmHg, about 9 mmHg, about 10 mmHg, about 11 mmHg, or about 12 mmHg.

In some embodiments, rather than automatically adjusting the shunt 205 in response to a determination that the pressure is outside the predefined range, the system 200 can provide an alert or notification to the patient, physician, or other user that the pressure is outside the predefined range. The alert can be generated by the external device 220 and can include an audio alert (e.g., an alarm), a visual alert, or the like. In some embodiments, the system 200 may provide a suggestion to the patient, physician, or other user to adjust the shunt 205. In other embodiments, the alert simply notifies the patient, physician, or other user that the pressure is outside of the predetermined range. A physician or other healthcare practitioner can then evaluate the patient and determine if any adjustment to the shunt 205 is necessary.

In some embodiments, the system 200 can be configured to reduce the risk of hypotony following the implantation of the shunt 205. Without being bound by theory, preventing and/or reducing flow through the shunt 205 immediately following implanting the shunt 205 is expected to reduce the risk of hypotony. Accordingly, when the shunt 205 is implanted, the flow control element on the shunt 205 can be in a first position in which the flow control element blocks and/or at least partially restricts flow through the shunt 205. The flow control element can be configured to increase flow through the shunt 205 after one or more criteria are met. For example, once the one or more criteria are met, the flow control element can transition from the first position to and/or toward a second position in which the flow control element does not block and/or only partially blocks flow through the shunt, thereby enabling increased flow through the shunt relative to the first position.

In some embodiments, the one or more criteria includes a predetermined pressure threshold. In such embodiments, the flow control element transitions from the first position to and/or toward the second position when the intraocular pressure exceeds the predetermined threshold. The predetermined threshold can be within a range of from about 18 mmHg to about 28 mmHg. For example, in some embodiments, the predetermined threshold is about 18 mmHg (e.g., 18 mmHg). In other embodiments, the predetermined threshold about 19 mmHg, about 20 mmHg, about 21 mmHg, about 22 mmHg, about 23 mmHg, about 24 mmHg, about 25 mmHg, about 26 mmHg, about 27 mmHg, or about 28 mmHg. In some embodiments, the flow control element can automatically transition from the first position to and/or toward the second position when the intraocular pressure exceeds the predetermined threshold. In other embodiments, an alert and/or instruction is provided to the patient and/or physician (e.g., via the external device 220) when the intraocular pressure exceeds the predetermined threshold. The alert can instruct the patient, physician, or other user to manually adjust the flow control element from the first position to and/or toward the second position.

In some embodiments, the one or more criteria includes a predetermined period of time. In such embodiments, the flow control element transitions from the first position to and/or toward the second position after a predetermined time period following implantation of the shunt has elapsed. The predetermined time period can be in a range of from about 1 day to about 3 months. In some embodiments, the predetermined time period can be in a range of about 3 weeks to about 7 weeks, or about 4 weeks to about 6 weeks. In some embodiments, the predetermined time period can be about 1 day, about 2 days, about 3 days, about 4 days, about 5 days, about 6 days, about 7 days, etc. The predetermined time period can also be about 1 week, about 2 weeks, about 3 weeks, about 4 weeks, etc. In some embodiments, the flow control element can automatically transition from the first position to and/or toward the second position after the predetermined period of time has elapsed. In other embodiments, an alert and/or instruction is provided to the patient and/or physician (e.g., via the external device 220) after the predetermined period of time has elapsed. The alert can instruct the patient, physician, or other user to manually adjust the flow control element form the first position to and/or toward the second position.

### C. Select Embodiments of Implantable Sensors and Associated External Devices

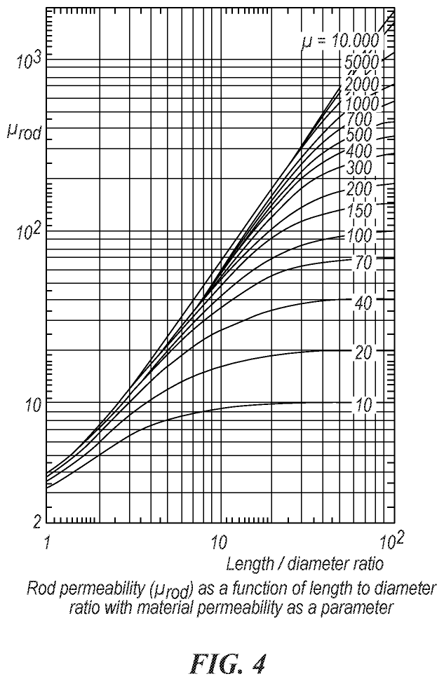

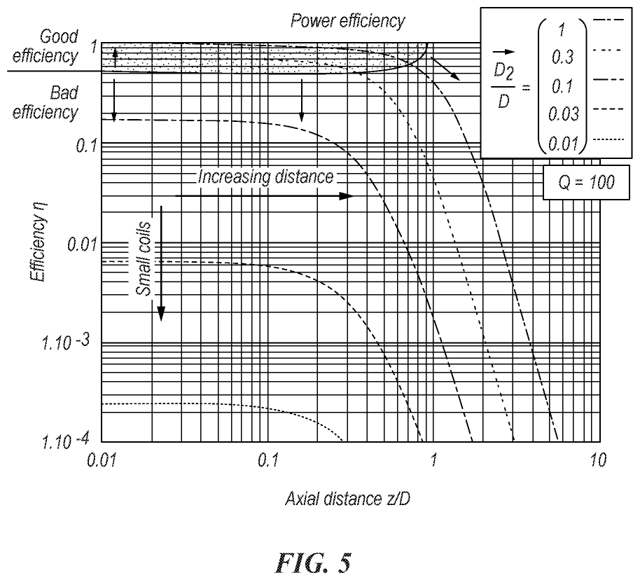

As provided above, the present technology includes implantable sensors for measuring one or more physiological parameters of a patient. The present technology also includes external devices that communicate with the implantable sensor and displays and/or analyzes the physiological parameters measured by the sensor. FIGS. 3A and 3B, for example, illustrate a sensor and an external device, respectively. However, as one skilled in the art will appreciate from the disclosure herein, the present technology can include many different types of sensors and external devices, and is not limited by the embodiments disclosed herein.

FIG. 3A is a schematic illustration of an implantable sensor 300 configured in accordance with embodiments of the present technology. The sensor 300 can include a sensing element 302, a signal amplifier 304, a microcontroller 306, a receiving antenna 308, and other associated electronics and/or circuitry. The sensing element 302 can be configured to measure a physiological parameter. For example, in some embodiments, the sensing element 302 is a pressure gauge, and the physiologic parameter is intraocular pressure or another parameter corresponding to intraocular pressure. The sensing element 302 can transmit a signal corresponding to the sensed data to the signal amplifier 304. The signal amplifier 304 can amplify the signal received from the sensing element 302 and transmit the amplified signal to the microcontroller 306. The microcontroller 306 can be configured to communicate with an external device (e.g., external device 220 described with respect to FIG. 2 and/or external device 350 described below with respect to FIG. 3B). In some embodiments, the microcontroller 306 is configured to communicate with the external device via frequency shift keying (“FSK”), on-off keying (“OOK”), Bluetooth, WiFi, or another suitable communication mechanism. Accordingly, the microcontroller 306 can transmit the amplified signal corresponding to the measured parameter to the external device for further processing, analysis, and/or display. The receiving antenna 308 can be configured to receive power for the sensor 300, as described in greater detail below.

In some embodiments, some or all of the electronic components on the sensor 300 can comprise standard miniature components configured to fit on a circuit board with a width of about 2 mm or less. For example, in some embodiments, the microcontroller 306 is an ATtiny20 or ATtiny102 microcontroller, which exist in miniature packages such as a 1.56×1.4 mm BGA or 2×3 mm UDFN package. In some embodiments, the signal amplifier 304 is a zero drift, low offset, low power opamp OPA330, which exists in a 1.1×0.8 mm BGA package. In some embodiments, the receiving antenna 308 is a ferrite antenna, such as a 2×5 mm or 2×10 mm coil with a miniature rectifier bridge. In a particular embodiment, the external device sends communication to the sensor 300 via OOK, and the sensor 300 sends communication to the external device via FSK. In such embodiment, if the frequency of the external magnetic field is 67 kHz, and the two modulation frequencies are 10 kHz and 20 kHz, the external device will see tones at 67 kHz+/−10 kHz and 67 kHz+/−20 kHz, respectively.

FIG. 3B is a schematic illustration of an external device 350 for communicating with the sensor 300 shown in FIG. 3A and configured in accordance with embodiments of the present technology. The external device 350 can be configured to receive data from the sensor 300, send power to the sensor 300, or both receive data from and send power to the sensor 300. The external device 350 can include a microcontroller 352, a receiver/demodulator 354, a transmitting antenna 358, and other associated electronics and/or circuitry. The receiver 354 can receive data from the sensor 300 (e.g., via FSK, OOK, Bluetooth, WiFi, etc.) and the microcontroller 352 can process the data. In some embodiments, the data processed by the microcontroller 352 can be transmitted to a display element (not shown in FIG. 3B) or another processor/computing device for further analysis (e.g., external device 220 described previously). The transmitting antenna 358 can transmit power to the sensor 300. For example, in some embodiments, the transmitting antenna 358 is a high Q coil driven by a class E amplifier, with a timer in the microcontroller 352 generating the driving waveform. In some embodiments, powering the sensor 300 and/or receiving data from the sensor 300 is done on demand and/or at predetermined time intervals.

In some embodiments, the microcontroller 306 and/or other implanted components of the sensor 300 are powered wirelessly (e.g., via an antenna). In other embodiments, the microcontroller 306 and/or other components of the sensor 300 can be powered via a wired connection, or a combination of a wired and wireless connection. For example, in some embodiments one or more features of the sensor 300 can be connected to a “hub” (e.g., positioned in the skull) via a wire. In such embodiments, the hub can be configured to transfer power to the sensor 300, as would be understood by one of skill in the art from the description herein.

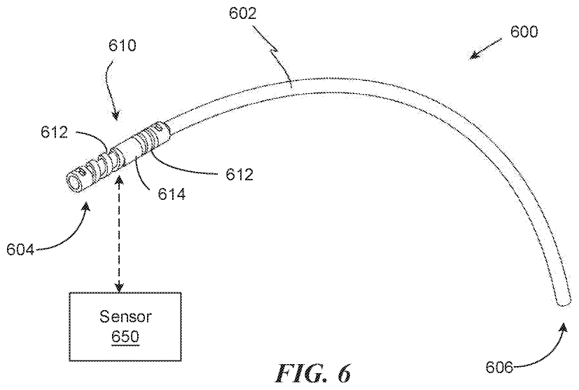

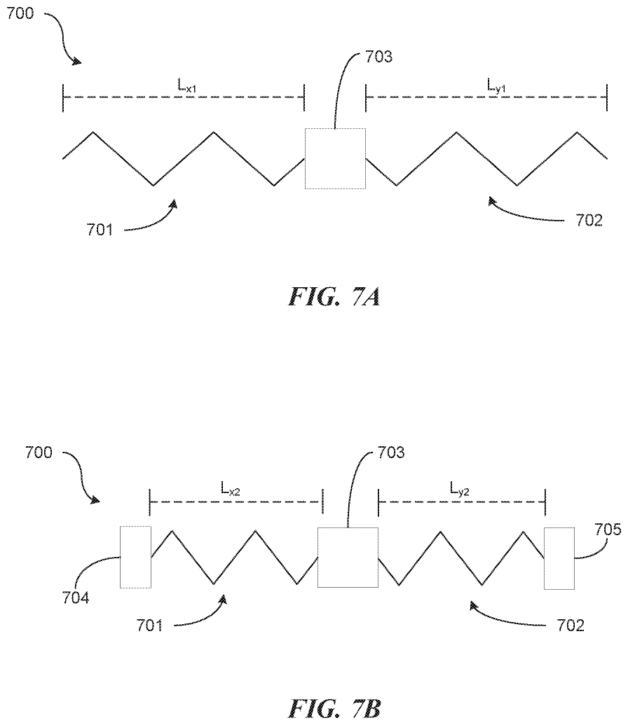

In some embodiments, the power needed to operate the sensor 300 is about 2 mW. For example, the power needed to bias the sensing element 202 can be around 1 mW, and the power needed for the operation of the microcontroller 206 can be about 1 mW. However, as one skilled in the art will understand form the disclosure herein, the power needed to operate the sensor 300 can vary according to the components used. For example, in some embodiments the receiving antenna 308 is a coil composed of a high permeability ferrite. In such embodiments, the receiving antenna 308 can be a 2×5 mm coil, a 2×10 mm coil, or the like. FIG. 4 is a graph illustrating the effective permeability of a ferrite antenna as a function of length to diameter. As illustrated, the effective permeability generally increases as the length to diameter ratio increases. For example, the effective permeability of a 2×5 mm coil is about 20, and the effective permeability of a 2×10 mm coil is about 60. For the 2×5 mm coil, the effective diameter of the receiving antenna 308 is 9 mm. For the 2×10 mm coil, the effective diameter of the receiving antenna 308 is 16 mm. The transmitting antenna 358 can also be a coil composed of a high permeability ferrite. In some embodiments, for example, the receiving antenna 308 is a 2×5 mm coil and the transmitting antenna 358 is a coil with a 10 cm diameter. When the 10 cm transmitting antenna is positioned about 10 cm away from the receiving antenna 308, the power transfer efficiency is about 1e-3, and the power requirement for the external device 350 is about 2 W or more. In some embodiments, the receiving antenna 308 is a 2×10 mm coil and the transmitting antenna 358 is a coil with a 50 cm diameter. When the 50 cm transmitting antenna is positioned about 50 cm away from the receiving antenna 308, the power transfer efficiency is about 1e-4, and the power requirement for the external device 350 greater than 20 W. FIG. 5 is a graph illustrating the power efficiency for an inductive power transfer system comprising loop inductors in dependence on their axial distance z with size ration as a parameter.

### D. Select Embodiments of Adjustable Flow Shunts

As provided above, in at least some embodiments the present technology provides adjustable flow shunts for treating a medical condition, such as glaucoma. The adjustable shunts described herein, such as shunt 205, can take any number of suitable forms, including those described in PCT Patent Application No. PCT/US2018/043158, filed Jul. 20, 2018, PCT Patent Application No. PCT/US2020/38549, filed Jun. 18, 2020, U.S. patent application Ser. No. 16/840,137, filed Apr. 14, 2020, U.S. Provisional Patent Application No. 62/929,608, filed Nov. 1, 2019, U.S. Provisional Patent Application No. 62/937,676, filed Nov. 19, 2019, U.S. Provisional Patent Application No. 62/937,680, filed Nov. 19, 2019, U.S. Provisional Patent Application No. 62/937,667, filed Nov. 19, 2019, U.S. Provisional Patent Application No. 62/965,117, filed Jan. 23, 2020, U.S. Provisional Patent Application No. 62/976,890, filed Feb. 14, 2020, U.S. Provisional Patent Application No. 62/978,210, field Feb. 18, 2020, U.S. Provisional Patent Application No. 62/981,411, filed Feb. 25, 2020, U.S. Provisional Patent Application No. 62/991,701, filed Mar. 19, 2020, U.S. Provisional Patent Application No. 63/003,594, filed Apr. 1, 2020, U.S. Provisional Patent Application No. 63/018,393, filed Apr. 30, 2020, and U.S. Provisional Patent Application No. 63/039,237, filed Jun. 15, 2020, the disclosures of which are all incorporated by reference herein in their entireties.

FIG. 6 illustrates an adjustable flow shunt 600 (“shunt 600”) configured in accordance with embodiments of the present technology. The shunt 600 includes an elongated drainage element 602 having an inflow region 604 and an outflow region 606. The inflow region 604 can have one or more inflow apertures (not shown) to allow fluid to flow into the drainage element 602. Likewise, the outflow region 606 can have one or more outflow apertures (not shown) to allow fluid to flow out of the drainage element 602. When the shunt 600 is implanted in a patient's eye, for example, the inflow region 604 can be in fluid communication with an anterior chamber of the eye and the outflow region 606 can be in fluid communication with a drainage location (e.g., a bleb space, a subconjunctival space, etc.). Aqueous can flow from the anterior chamber to the drainage location through the drainage element 602.

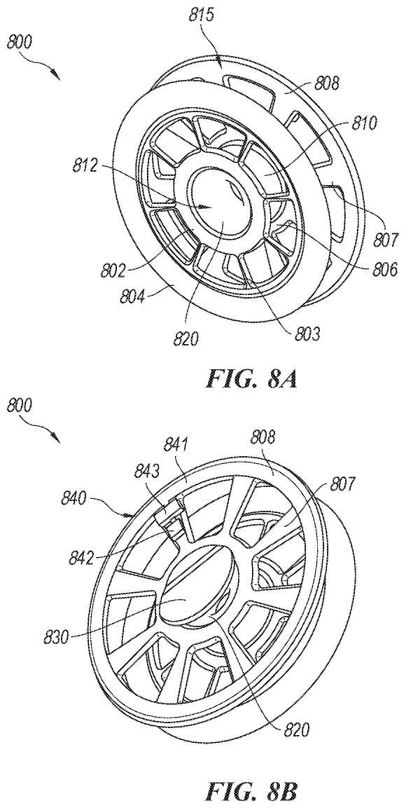

The shunt 600 further includes an actuation assembly 610 positioned adjacent the inflow region 604. However, although illustrated as being coupled to the inflow region 604, in other embodiments the actuation assembly 610 can be coupled to the outflow region 606 of the shunt 600. The actuation assembly 610 can include actuation elements 612 and a flow control element 614. The actuation assembly 610 can be configured to control the flow of fluid through the shunt 600, such as, for example, by selectively blocking and/or unblocking (or partially block and/or partially unblocking) the one or more inflow apertures at the inflow region 604 using the flow control element 614. Additional details of actuation assemblies for use with adjustable shunts are described below with respect to FIGS. 7A-7D. In some embodiments, the actuation assembly 610 is the same as the actuation assembly 700 described in detail below, although in other embodiments the actuation assembly 610 can be a modified version of the actuation assembly 700.

In some embodiments, the shunt 600 can be operably coupled to a sensor 650. The sensor 650 can be any sensor previously described, such as a pressure gauge. The sensor 650 may be physically coupled to the shunt 600 (e.g., carried by, tethered to, etc.) and/or wirelessly coupled to the shunt 600. Together, the sensor 650 and the shunt 600 can perform any of the operations described herein (with or without the addition of one or more external devices (not shown)). For example, in at least some embodiments the actuation assembly 610 is configured to adjust a position of the flow control element 614 based at least in part on one or more measurements taken by the sensor 650.

FIGS. 7A-7D illustrate an embodiment of an actuation assembly 700 for use with an adjustable flow shunt and configured in accordance with select embodiments of the present technology. The actuation assembly 700 includes a flow control element 703 that is configured to interface with an aperture (e.g., the inflow aperture on the shunt 600) of a shunt (not shown in FIGS. 7A-7D). As described below, the flow control element 703 is moveable between a plurality of positions relative to a shunt to progressively block and/or progressively unblock the aperture. By further blocking the aperture, the flow control element 703 reduces flow through the shunt. By further unblocking the aperture, the flow control element 703 increases flow through the shunt.

Referring collectively to FIGS. 7A-7D, the actuation assembly 700 can include a first actuation element 701 and a second actuation element 702. The first actuation element 701 can extend between the flow control element 703 and a first anchoring element 704. The second actuation element 702 can extend between the flow control element 703 and a second anchoring element 705. The first anchoring element 704 and the second anchoring element 705 can be secured to a generally static component of the shunt (not shown). In other embodiments, the first anchoring element 704 and/or the second anchoring element 705 can be omitted and the first actuation element 701 and/or the second actuation element 702 can be secured directly to a portion of the device or system for shunting fluid (not shown). In any of these embodiments, selectively modifying fluid flow through the shunt by moving the flow control element 703 occurs without damaging or otherwise negatively affecting tissue of the patient.

The first actuation element 701 and the second actuation element 702 can be composed of a shape memory material, such as a shape memory alloy (e.g., nitinol). Accordingly, the first actuation element 701 and the second actuation element 702 can be transitionable between a first state (e.g., a martensitic state, a R-phase, etc.) and a second state (e.g., a shape memory state, an austenitic state, etc.). In the first state, the first actuation element 701 and the second actuation element 702 may be deformable (e.g., plastic, malleable, compressible, expandable, etc.). In the second state, the first actuation element 701 and the second actuation element 702 may have a preference toward a specific original shape (e.g., geometry, length, and/or or dimension). The first actuation element 701 and the second actuation element 702 can be transitioned between the first state and the second state by applying energy (e.g., heat) to the actuation elements to heat the actuation elements above a transition temperature. In some embodiments, the transition temperature for both the first actuation element 701 and the second actuation element 702 is above an average body temperature. Accordingly, both the first actuation element 701 and the second actuation element 702 are typically in the deformable first state when the actuation assembly 700 is implanted in the body until they are heated (e.g., actuated).

If an actuation element (e.g., the first actuation element 701) is deformed relative to its original shape while in the first state, heating the actuation element (e.g., the first actuation element 701) above its transition temperature causes the actuation element to transition to the second state and therefore transition from the deformed shape to the original shape. Heat can be applied to the actuation elements via an energy source positioned external to the body (e.g., a laser), RF heating, resistive heating, or the like. In some embodiments, the first actuation element 701 can be selectively heated independently of the second actuation element 702, and the second actuation element 702 can be selectively heated independently of the first actuation element 701.

Referring to FIG. 7A, the first actuation element 701 and the second actuation element 702 are shown in a state before being secured to the first and second anchoring elements. In particular, the first actuation element 701 and the second actuation element 702 are in their unbiased original shapes (e.g., memory shape, heat set shape, etc.). In the illustrated embodiment, the first actuation element 701 has an original shape having a length Lₓ₁, and the second actuation element 702 has an original shape having a length L_{y1}. In some embodiments, Lₓ₁ is equal to L_{y1}. In other embodiments, Lₓ₁ is less than or greater than (i.e., not equal to) L_{y1}.

FIG. 7B illustrates the actuation assembly 700 in a first (e.g., composite) configuration after the first actuation element 701 has been secured to the first anchoring element 704, and the second actuation element 702 has been secured to the second anchoring element 705. In the first configuration, both the first actuation element 701 and the second actuation element 702 are at least partially deformed relative to their original shape. For example, the first actuation element 701 is compressed (e.g., shortened) relative to its original shape (FIG. 7A) such that it assumes a second length Lₓ₂ that is less than the first length Lₓ₁. Likewise, the second actuation element 702 is also compressed (e.g., shortened) relative to its original shape (FIG. 7A) such that it assumes a second length L_{y2} that is less than the first length L_{y1}. In the illustrated embodiment, Lₓ₁ is equal to L_{y1}, although in other embodiments Li can be less than or greater than (i.e., not equal to) L_{y1}. In other embodiments, the first actuation element 701 and/or the second actuation element 702 are stretched (e.g., lengthened) relative to their original shape before being secured to the anchoring elements. For example, in some embodiments, the first actuation element 701 is compressed (e.g., shortened) relative to its original shape and the second actuation element 702 is stretched (e.g., lengthened) relative to its original shape. In some embodiments, only one of the actuation elements (e.g., the first actuation element 701) is deformed relative to its original shape, and the other actuation element (e.g., the second actuation element 702) retains its original shape.

FIG. 7C illustrates the actuation assembly 700 in a second configuration different than the first configuration. In particular, in the second configuration, the actuation assembly 700 has been actuated relative to the first configuration shown in FIG. 7B to transition the first actuation element 701 from the first (e.g., martensitic) state to the second (e.g., austenitic) state. Because the first actuation element 701 was deformed (e.g., compressed) relative to its original shape while in the first configuration, heating the first actuation element 701 above its transition temperature causes the first actuation element 701 to assume its original shape having a length Li (FIG. 7A). As described above, the first anchoring element 704 and the second anchoring element 705 are fixedly secured to a generally static structure (e.g., such that a distance between the first anchoring element 704 and the second anchoring element 705 does not change during actuation of the first actuation element 701). Accordingly, as the first actuation element 701 increases in length toward its original shape, the second actuation element 702, which is unheated and therefore remains in the generally deformable (e.g., martensitic) state, is further compressed to a length L_{y3} that is less than L_{y1} and L_{y2}. In the illustrated embodiment, this moves the flow control element 703 away from the first anchoring element 704 and toward the second anchoring element 705.

FIG. 7D illustrates the actuation assembly 700 in a third configuration different than the first configuration and the second configuration. In particular, in the third configuration the actuation assembly 700 has been actuated relative to the second configuration shown in FIG. 7C to transition the second actuation element 702 from the first (e.g., martensitic) state to the second (e.g., austenitic) state. Because the second actuation element 702 was deformed (e.g., compressed) relative to its original shape while in the second configuration, heating the second actuation element 702 above its transition temperature causes the second actuation element 702 to assume its original shape having a length L_{y1} (FIG. 7A). As described above, the first anchoring element 704 and the second anchoring element 705 are fixedly secured to a generally static structure (e.g., such that the distance between the first anchoring element 704 and the second anchoring element 705 does not change during actuation of the second actuation element 702). Accordingly, as the second actuation element 702 increases in length toward its original shape, the first actuation element 701, which is unheated and therefore remains in the generally deformable (e.g., martensitic) state, is further deformed (e.g., compressed) relative to its original shape to a length Lₓ₃ that is less than Lₓ₁ and Lₓ₂. In the illustrated embodiment, this moves the flow control element 703 away from the second anchoring element 705 and toward the first anchoring element 704 (e.g., generally opposite the direction the flow control element 703 moves when the first actuation element 701 is actuated).

The actuation assembly 700 can be repeatedly transitioned between the second configuration and the third configuration. For example, the actuation assembly 700 can be returned to the second configuration from the third configuration by heating the first actuation element 701 above its transition temperature once the second actuation element 702 has returned to the deformable first state (e.g., by allowing the second actuation element 702 to cool below the transition temperature). Heating the first actuation element 701 above its transition temperature causes the first actuation element 701 to assume its original shape, which in turn pushes the flow control element 703 back toward the second anchoring element 705 and transitions the actuation assembly 700 to the second configuration (FIG. 7C). Accordingly, the actuation assembly 700 can be selectively transitioned between a variety of configurations by selectively actuating either the first actuation element 701 or the second actuation element 702. After actuation, the actuation assembly 700 can be configured to substantially retain the given configuration until further actuation of the opposing actuation element. In some embodiments, the actuation assembly 700 can be transitioned to intermediate configurations between the second configuration and the third configuration (e.g., the first configuration) by heating a portion of the first actuation element 701 or the second actuation element 702.

As provided above, heat can be applied to the actuation elements via an energy source positioned external to the body (e.g., a laser), RF heating, resistive heating, or the like. In some embodiments, an external device (e.g., external device 220) directs the energy source to heat the one or more of the actuation elements based on readings from one or more sensors (e.g., sensor 210). In other embodiments, a user (e.g., a physician) operates the energy source to heat one or more of the actuation elements based on readings from one or more sensors. In some embodiments, the first actuation element 701 can be selectively heated independently of the second actuation element 702, and the second actuation element 702 can be selectively heated independently of the first actuation element 701. For example, in some embodiments, the first actuation element 701 is on a first electrical circuit and/or responds to a first frequency range for selectively and resistively heating the first actuation element 701 and the second actuation element 702 is on a second electrical circuit and/or responds to a second frequency range for selectively and resistively heating the second actuation element 702. As described in detail above, selectively heating the first actuation element 701 moves the flow control element 703 in a first direction and selectively heating the second actuation element 702 moves the flow control element 703 in a second direction generally opposite the first direction. The actuation assembly 700 can therefore be adjusted to achieve any of the operations described herein with respect to adjustable shunts.

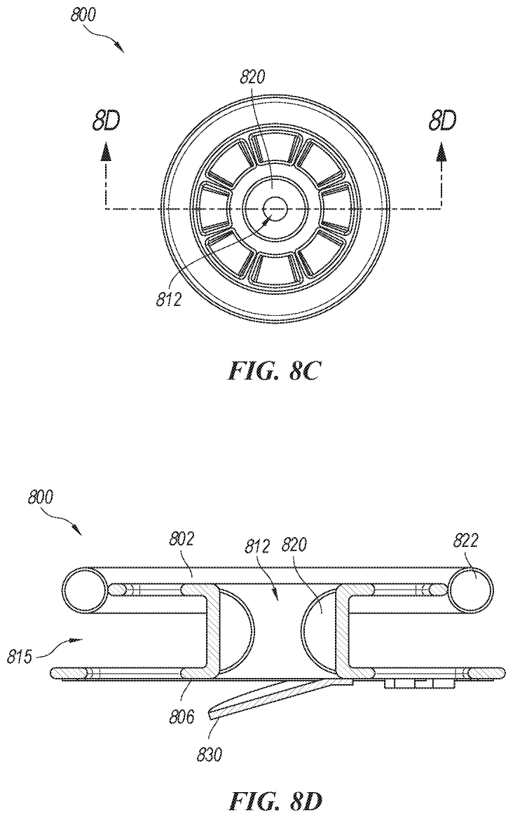

FIGS. 8A-8B illustrate another adjustable flow shunt 800 (“shunt 800”) configured in accordance with embodiments of the present technology. In some embodiments, the shunt 800 can be configured to treat a patient with heart failure, such as by shunting fluid between a left atrium (LA) and a right atrium (RA) of the patient's heart. Referring first to FIG. 8A, which is a partially isometric view of the shunt 800, the shunt 800 can include a shunting or tubular element 810 having a lumen 812 extending therethrough. When the shunt 800 is implanted in a patient (e.g., within a heart and across a septal wall), the lumen 812 can fluidly connect a first body region (e.g., the LA) and a second body region (e.g., the RA) to shunt fluid (e.g., blood) therebetween. As described in greater detail below with respect to FIGS. 8C and 8D, a flow control element 820 can be placed within the tubular element 810 to control the flow of fluid between the first body region and the second body region.

The shunt 800 can be secured across the septal wall or other anatomical structure using one or more anchoring elements, such as flanges. In the illustrated embodiment, for example, the shunt 800 includes a first flange 802 having a plurality of first spokes 803 and a first ring 804. The shunt 800 also includes a second flange 806 having a plurality of second spokes 807 and a second ring 808. In other embodiments, the first flange 802 and/or the second flange 806 extend radially outward as a circular plate-like structure, and the first spokes 803 and the second spokes 807 are omitted. The first flange 802 and the second flange 806 can be at least partially spaced apart to create a gap 815. The gap 815 can be configured to receive native tissue (e.g., a portion of the septal wall). Accordingly, when the shunt 800 is implanted within a heart, the first flange 802 can reside on a LA side of the septal wall, the second flange 806 can reside on a RA side of the septal wall, and a portion of the septal wall can be disposed in the gap 815 between the first flange 802 and the second flange 806, thereby securing the shunt 800 in place. In some embodiments, the first flange 802 and the second flange 806 can be transitionable between a generally low-profile delivery configuration and an expanded deployed configuration. For example, in some embodiments at least some aspects of the first flange 802 and the second flange 806 are inflatable such that after delivery of the shunt 800, the first flange 802 and the second flange 806 can be inflated to expand from the low-profile delivery configuration to the deployed configuration, thereby securing the shunt 800 in position. In some embodiments, the shunt 800 may have additional or alternative anchoring mechanisms to secure the shunt 800 in position.

FIG. 8B is a partially isometric view of the shunt 800 from an outflow side of the shunt 800. As illustrated in FIG. 8B, the shunt 800 can optionally include a valve or flap 830 that can close to block blood flow through the lumen 812. The flap 830 can be a one-way valve that permits fluid flow in a first direction (e.g., blood flow from the LA to the RA) and prevents and/or reduces fluid flow in a second direction (e.g., blood flow from the RA to the LA). Accordingly, the flap 830 can reduce the risk of backflow through the lumen 812 when the shunt 800 is implanted in the septal wall or another location. In some embodiments, the flap 830 is omitted and flow through the lumen 812 is controlled through inflation and deflation of the flow control element 820, as described in greater detail below.

FIG. 8C is a front view of the shunt 800, and FIG. 8D is a cross-section view of the shunt 800 taken along the line 8D-8D indicated in FIG. 8C. As illustrated, the flow control element 820 can have a generally toroidal shape that, in at least some configurations, occupies at least a portion of the lumen 812. Accordingly, the flow control element 820 can at least partially block the lumen 812. In some embodiments, the flow control element 820 is an at least partially flexible (e.g., expandable and/or compressible) structure (e.g., a bladder, cavity, balloon, etc.) that can hold a fluid (e.g., saline, silicon oil, hydrogel) or a gas (e.g., air). Accordingly, the flow control element 820 can inflate (e.g., fill with liquid or gas) and/or deflate (e.g., unfill) to change the shape and or size of the lumen 812. The flow control element 820 can also be referred to as an “expandable flow restrictor” or an “expandable member.” As described in detail below, the flow control element 820 may fill and/or unfill depending on, for example, the pressure differential between the environment surrounding the flow control element 820 (e.g., the lumen 812) and the environment surrounding another bladder or reservoir (e.g., a reservoir 822) in fluid communication with the flow control element 820.

As noted above, the shunt 800 can also include a reservoir 822 fluidly coupled to the flow control element 820. Accordingly, as described in detail below, the fluid or gas can be routed between the reservoir 822 and the flow control element 820. In some embodiments, the reservoir 822 is at least partially flexible (e.g., expandable and/or compressible). Accordingly, the reservoir 822 can inflate (e.g., fill with liquid or gas) and/or deflate (e.g., unfill) based on the relative presence or absence of gas or fluid in the reservoir 822. In other embodiments, the reservoir 822 does not change in shape or size as fluid or gas flows into and/or out of the reservoir 822. In some embodiments, the reservoir 822 can be positioned on or within the first flange 802, on or within the second flange 806, on or within another suitable structure of the shunt 800, or on or within a combination of structures of the shunt 800. In some embodiments, the reservoir 822 is positioned within a housing formed by the first flange 802 or the second flange 806 such that the pressure exerted on the reservoir 822 is generally constant. In other embodiments, the reservoir 822 may be at least partially exposed to a heart chamber (e.g., a LA or an RA), and the pressure exerted on the reservoir 822 is determined at least in part by the pressure in the heart chamber.

Fluid or gas can flow between the reservoir 822 and the flow control element 820 (and vice versa) to fill (e.g., inflate) and/or unfill (e.g., deflate) the flow control element 820 and the reservoir 822. Filling and/or unfilling the flow control element 820 changes the size and/or shape of the lumen 812 and can accordingly change the flow resistance and/or the flow of blood through the lumen 812. For example, the flow control element 820 inflates as fluid flows into the flow control element 820 and out of the reservoir 822, thereby reducing the size of the lumen 812 (and the flow of blood between the first body region and the second body region). The flow control element 820 deflates as fluid flows out of the flow control element 820 and into the reservoir 822, thereby increasing the size of the lumen 812 (and the flow of blood between the first body region and the second body region). In some embodiments, and as described below, the flow of fluid between the reservoir 822 and the flow control element 820 can be passively controlled based on, among other things, a pressure differential between the first body region and the second body region.

In some embodiments, the shunt 800 can be operably coupled to a sensor 840. The sensor 840 can be any sensor previously described, such as a pressure gauge. In the illustrated embodiment, the sensor 840 includes a pressure gauge 842, a data antenna 841, and a control circuit 843. The sensor 840 can be configured to measure one or more physiological parameters surrounding the shunt 800, such as left atrial pressure and/or right atrial pressure. In the illustrated embodiment, the sensor 840 is illustrated on the outflow side (e.g., the RA side) of the shunt 800, although in other embodiments the sensor 840 can be on an inflow side (e.g., LA side) of the shunt 800. In yet other embodiments, the shunt 800 includes a sensor 840 on both the inflow side of the shunt 800 and the outflow side of the shunt 800. Although only illustrated as including one sensor 840, the shunt 800 can have multiple sensors (e.g., arranged along a perimeter of the first ring 804 and/or the second ring 808). The data antenna 841 can communicate data to and or from the sensor 840. For example, the data antenna 841 may be able to communicate with an external device or controller (e.g., external device 220 in FIG. 2). The control circuit 843 can control power delivered from the data antenna 841 and/or signals received from the pressure sensor 842 and delivered to the data antenna 841. Together, the sensor 850 and the shunt 800 can perform any of the operations described herein (with or without the addition of one or more external devices (not shown)). For example, in some embodiments the flow of fluid between the reservoir 822 and the flow control element 820 can be based on one or more measurements taken by the sensor 850 such that the flow of blood through the shunt is based at least in part on the parameters measured by the sensor 850.

### Examples

Several aspects of the present technology are set forth in the following examples:

1. A glaucoma treatment system, the system comprising:
an adjustable flow shunt having (a) an inflow end region, (b) an outflow end region, (c) a drainage tube fluidly connecting the inflow end region and outflow end region, and (d) a flow control element configured to control fluid flow through the shunt,
wherein, when implanted into an eye of a patient, the inflow end region is in fluid communication with an anterior chamber of the eye, the outflow end region is in fluid communication with a subconjunctival space, and the device is configured to direct the flow of aqueous humor from the anterior chamber to the subconjunctival space; and
an implantable pressure sensor operably coupled to the adjustable flow shunt, wherein the pressure sensor is configured to detect a pressure value indicative of an intraocular pressure,
wherein, when the detected intraocular pressure is outside a predetermined range of intraocular pressure, the flow control element is adjusted to change flow through the shunt.

2. The glaucoma treatment system of example 1, further comprising an external device configured to be external to the patient, wherein the external device includes a processor, and wherein the external device and implantable sensor are configured to wirelessly communicate such that the processor receives the detected pressure value.

3. The glaucoma treatment system of example 2 wherein—
the implantable pressure sensor includes a receive antenna,
the external device includes a power transmitter, and
the external device is configured to charge the implantable pressure sensor by transmitting power to the receive antenna via the power transmitter.

4. The glaucoma treatment system of example 3 wherein the external device is operably coupled to a display element, and wherein the display element is configured to display the pressure value.

5. A method of treating glaucoma using an adjustable flow shunt implanted in a human eye to shunt aqueous humor from an anterior chamber of the eye to a bleb space, the adjustable flow shunt having a flow control element configured to control fluid flow therethrough, the method comprising:
measuring a physiological parameter indicative of an intraocular pressure via an implantable pressure sensor;
determining, based at least in part on the measured physiological parameter, the intraocular pressure; and
if the intraocular pressure is outside a predetermined range of intraocular pressures, adjusting the flow control element to alter the fluid flow therethrough.

6. The method of claim 5 wherein the predetermined range of intraocular pressures is between about 12 mmHg and 22 mmHg.

7. The method of example 5 wherein the predetermined range of intraocular pressures is between about 10 mmHg and 25 mmHg.

8. The method of example 5 wherein the predetermined range of intraocular pressures is between about 5 mmHg and 25 mmHg.

9. A method of reducing the risk of hypotony during glaucoma treatment, the method comprising:
implanting an adjustable flow shunt into the eye of a patient such that an inflow region of the shunt is in fluid communication with an anterior chamber of a human eye and an outflow end region is in fluid communication with a bleb space, wherein the adjustable flow shunt has a flow control element configured to control the flow of fluid from the anterior chamber to the bleb space, and wherein, when implanted, the flow control element has a first position;
measuring the intraocular pressure via an implanted pressure sensor; and
moving the flow control element to a second position (a) when the measured intraocular pressure exceeds a predetermined threshold, and/or (b) after a predetermined time-period following implantation has elapsed, wherein the second position enables increased fluid flow between the anterior chamber and the bleb space than the first position.

10. The method of example 9 wherein the predetermined time-period is about one week.

11. The method of example 9 wherein the predetermined time-period is about two weeks.

12. The method of any of examples 9-11, wherein the flow control element is moved to the second position when (a) the measured intraocular pressure exceeds the predetermined threshold, and (b) the predetermined time-period following implantation has elapsed.

13. A computer-implemented method of altering fluid flow through an adjustable flow shunt in the treatment of glaucoma, the shunt having a flow control element controlling fluid flow therethrough, the computer-implanted method comprising:
directing a pressure sensor implanted in an eye to measure a pressure value indicative of an intraocular pressure;
receiving, via the pressure sensor, the pressure value indicative of the intraocular pressure; and
determining, based at least in part on the received pressure value, whether to adjust a position of the flow control element, wherein adjusting the position of the flow control element selectively adjusts the fluid flow through the shunt.

14. The computer-implemented method of example 13, further comprising displaying, via a display element, the pressure value.

15. The computer-implemented method of example 13 or 14, further comprising automatically adjusting the position of the flow control element based at least in part on the received pressure value.

16. The computer-implemented method of example 13 or 14, further comprising indicating to a user whether to adjust the flow control element.

17. An adjustable flow shunt for treating glaucoma in a human patient, the shunt comprising:
an elongated outflow drainage tube having a proximal inflow region configured for fluid communication with an anterior chamber of an eye and a distal outflow region configured for fluid communication with a bleb;
a flow control assembly at either the proximal inflow region or the distal outflow region, wherein the flow control assembly is configured to selectively control the flow of fluid through the drainage tube;
a pressure sensor adjacent the proximal inflow region, wherein the pressure sensor is configured to measure an intraocular pressure; and
a wire coupled to the pressure sensor and configured to transmit a signal indicative of the intraocular pressure.

18. An adjustable flow shunt for treating glaucoma in a human patient, the shunt comprising:
an elongated outflow drainage tube having a proximal inflow region configured for fluid communication with an anterior chamber of an eye and a distal outflow region configured for fluid communication with a bleb;
a flow control assembly at either the proximal inflow region or the distal outflow region, wherein the flow control assembly is configured to selectively control the flow of fluid through the drainage tube;
a pressure sensor adjacent the proximal inflow region, wherein the pressure sensor is configured to measure an intraocular pressure; and
an antenna operably coupled to the pressure sensor, wherein the antenna is configured to receive power from an external power source and provide energy to the pressure sensor.

19. A method of evaluating a patient having glaucoma, the method comprising:
measuring, via an implantable pressure sensor, an intraocular pressure in an eye of the patient;
transmitting the measured intraocular pressure to an external device; and
evaluating, based at least in part on the measured intraocular pressure, the patient's condition.

20. The method of example 19, further comprising generating a notification if the intraocular pressure exceeds a predetermined threshold.

21. The method of example 20 wherein the predetermined threshold is about 25 mmHg.

22. The method of any one of examples 19-21, further comprising generating a notification if the intraocular pressure falls below a predetermined threshold.

23. The method of example 22, wherein the predetermined threshold is about 10 mmHg.

24. The method of any of examples 19-23, further comprising generating a notification if a rate of change of the measured intraocular pressure exceeds a predetermined threshold.

25. A method of evaluating a patient having glaucoma, the method comprising:
periodically measuring an intraocular pressure in an eye of the patient at first time intervals for a first period of time following implantation of a shunt;
after expiration of the first period of time, periodically measuring the intraocular pressure in the eye of the patient at second time intervals for a second period of time, wherein the second time intervals are greater than the first time intervals.

26. A pressure monitoring system for use with an adjustable flow shunt for treating glaucoma, the system comprising:
an implantable pressure sensor configured to detect and transmit a pressure value indicative of an intraocular pressure within a human eye; and
an external device wirelessly coupled to the implantable pressure sensor and configured to receive and display the transmitted pressure value,
wherein the pressure value is used to determine whether to change the resistance of the adjustable flow shunt to allow a greater or lesser fluid flow therethrough.

27. A system for treating glaucoma, the system comprising:
an adjustable flow shunt having an inflow region, an outflow region, and a flow control element configured to control the flow of fluid through the adjustable flow shunt, wherein the adjustable flow shunt is configured to be implanted into an eye of a patient such that the inflow region of the shunt is in fluid communication with an anterior chamber of the eye and the outflow region is in fluid communication with a target drainage location; and
an implantable sensor configured to measure an intraocular pressure of the eye,
wherein—
   the flow control element is in a first position when the adjustable flow shunt is implanted, and
   the flow control element is configured to transition between the first position and a second, different position that enables increased fluid flow between the anterior chamber and the bleb space relative to the first position (a) when the measured intraocular pressure exceeds a predetermined threshold, (b) after a predetermined time-period following implantation has elapsed, or both (a) and (b).

28. The system of example 27 wherein the flow control element is configured to transition to the second position when the measured intraocular pressure exceeds the predetermined threshold.

29. The system of example 28 wherein the predetermined threshold is between about 18 mmHg and about 28 mmHg.

30. The system of example 27 wherein the flow control element is configured to transition to the second position after the predetermined time-period following implantation has elapsed.

31. The system of example 30 wherein the predetermined time-period is between about one week and about eight weeks.

32. The system of example 30 wherein the predetermined time-period is between about four weeks and about six weeks.

33. The system of example 27 wherein the flow control element is configured to transition to the second position only once both (a) the measured intraocular pressure exceeds the predetermined threshold and (b) the predetermined time-period following implantation has elapsed.

34. The system of any of examples 27-33 wherein the flow control element is configured to automatically transition to the second position.

35. A system for treating glaucoma, the system comprising:
an adjustable flow shunt having an inflow region, an outflow region, and a flow control element configured to control fluid flow through the shunt, wherein, when implanted into an eye—
   the inflow region is in fluid communication with an anterior chamber of the eye, the outflow end region is in fluid communication with a drainage location, and the device directs the flow of aqueous from the anterior chamber to the drainage location, and
   the flow control element is transitionable between at least a first position enabling a first amount of aqueous to flow through the shunt and a second position enabling a second amount of aqueous different than the first amount to flow through the shunt; and
an implantable pressure sensor configured to intermittently measure a pressure value indicative of an intraocular pressure at a predetermined time interval;
wherein the system is configured such that—
   if the determined pressure value exceeds a first predetermined threshold, (i) the system moves the flow control element toward the second position, or (ii) the system generates a notification instructing a user to move the flow control element toward the second position, and
   if the determined pressure value falls below a second predetermined threshold, (iii) the system moves the flow control element toward the first position, or (iv) the system generates a notification instructing a user to move the flow control element toward the first position.

36. The system of example 35 wherein the flow control element has a plurality of discrete positions between the first position and the second position, and wherein each of the plurality of discrete positions enables a different amount of aqueous to flow through the shunt.

37. The system of example 35 or 36 wherein the predetermined time interval is daily.

38. The system of example 35 or 36 wherein the predetermined time interval is weekly.

39. The system of any of examples 35-38 wherein the first predetermined threshold is between about 18 mmHg and about 28 mmHg.

40. The system of any of examples 35-39 wherein the second predetermined threshold is between about 5 mmHg and about 12 mmHg.

41. The system of any of examples 35-40 wherein the sensor is physically coupled to the shunt.

42. The system of any of examples 35-40 wherein the sensor is wirelessly coupled to the shunt.

43. The system of any of examples 35-42 wherein if the determined pressure value exceeds the first predetermined threshold, the system automatically moves the flow control element toward the second position, and wherein if the determined pressure value falls below the second predetermined threshold, the system automatically moves the flow control element toward the first position.

44. A computer-implemented method of altering fluid flow through an adjustable flow shunt in the treatment of glaucoma, the shunt having a flow control element controlling fluid flow through the shunt, the computer-implanted method comprising:
directing a pressure sensor implanted in an eye to measure a pressure value indicative of an intraocular pressure;
receiving, from the pressure sensor, the pressure value indicative of the intraocular pressure; and
determining, based at least in part on the received pressure value, whether to adjust a position of the flow control element to adjust the fluid flow through the shunt.

45. The computer-implemented method of example 44, further comprising displaying, via a display element, the pressure value.

46. The computer-implemented method of example 44 or 45 wherein determining whether to adjust a position of the flow control element comprises automatically determining whether to adjust a position of the flow control element based on one or more criteria.

47. The computer-implemented method of example 46 wherein the one or more criteria includes a pressure range.

48. The computer-implemented method of example 46 or 47, further comprising automatically directing an actuator to adjust the position of the flow control element based on a determination to adjust the flow control element.

49. The computer-implemented method of example 46 or 47, further comprising instructing a user to adjust the flow control element based on a determination to adjust the flow control element.

50. A system for draining fluid from a first body region to a second body region, the system comprising:
an adjustable flow shunt having an inflow region, an outflow region, and a flow control element configured to control fluid flow through the shunt, wherein, when implanted into the patient—
   the inflow region is in fluid communication with the first body region, the outflow end region is in fluid communication with the second body region, and the device directs the flow of fluid from the first body region to the second body region, and
   the flow control element is transitionable between at least a first position enabling a first amount of fluid to flow through the shunt and a second position enabling a second amount of fluid different than the first amount to flow through the shunt; and
an implantable pressure sensor configured to intermittently measure a pressure value indicative of a pressure in the first body region at a predetermined time interval;
wherein the system is configured such that—
   if the determined pressure value exceeds a first predetermined threshold, (i) the system moves the flow control element toward the second position, or (ii) the system generates a notification instructing a user to move the flow control element toward the second position, and
   if the determined pressure value falls below a second predetermined threshold, (iii) the system moves the flow control element toward the first position, or (iv) the system generates a notification instructing a user to move the flow control element toward the first position.

51. The system of example 50 wherein the flow control element has a plurality of discrete positions between the first position and the second position, and wherein each of the plurality of discrete positions enables a different amount of fluid to flow through the shunt.

52. The system of example 50 or 51 wherein the predetermined time interval is daily.

53. The system of example 50 or 51 wherein the predetermined time interval is weekly.

54. The system of any of examples 50-53 wherein the first predetermined threshold is between about 18 mmHg and about 28 mmHg.

55. The system of any of examples 50-54 wherein the second predetermined threshold is between about 5 mmHg and about 12 mmHg.

56. The system of any of examples 50-55 wherein the sensor is physically coupled to the shunt.

57. The system of any of examples 50-57 wherein the sensor is wirelessly coupled to the shunt.

58. The system of any of examples 50-57 wherein if the determined pressure value exceeds the first predetermined threshold, the system automatically moves the flow control element toward the second position, and wherein if the determined pressure value falls below the second predetermined threshold, the system automatically moves the flow control element toward the first position.

59. A system for draining fluid from a first body region to a second body region, the system comprising:
an adjustable flow shunt having an inflow region, an outflow region, and a flow control element configured to control the flow of fluid through the adjustable flow shunt, wherein the adjustable flow shunt is configured to be implanted into a patient such that the inflow region of the shunt is in fluid communication with the first body region and the outflow region is in fluid communication with the second body region; and
an implantable sensor configured to measure a pressure in the first body region,
wherein—
   the flow control element is in a first position when the adjustable flow shunt is implanted, and
   the flow control element is configured to transition from the first position to a second, different position that enables increased fluid flow between the first body region and the second body region relative to the first position (a) when the measured pressure exceeds a predetermined threshold, (b) after a predetermined time-period following implantation has elapsed, or both (a) and (b).

60. The system of example 59 wherein the flow control element is configured to transition to the second position when the measured pressure exceeds the predetermined threshold.

61. The system of example 60 wherein the predetermined threshold is between about 18 mmHg and about 28 mmHg.

62. The system of example 59 wherein the flow control element is configured to transition to the second position after the predetermined time-period following implantation has elapsed.

63. The system of example 62 wherein the predetermined time-period is between about one week and about eight weeks.

64. The system of example 62 wherein the predetermined time-period is between about four weeks and about six weeks.

65. The system of example 59 wherein the flow control element is configured to transition to the second position only once both (a) the measured pressure exceeds the predetermined threshold and (b) the predetermined time-period following implantation has elapsed.

66. The system of any of examples 59-65 wherein the flow control element is configured to automatically transition to the second position.

### CONCLUSION

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, any of the features of the intraocular shunts described herein may be combined with any of the features of the other intraocular shunts described herein and vice versa. Moreover, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions associated with intraocular shunts have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A system for treating glaucoma, the system comprising:
an adjustable flow shunt having an inflow region, an outflow region, and a flow control element configured to control the flow of fluid through the adjustable flow shunt, wherein the adjustable flow shunt is configured to be implanted into an eye of a patient such that the inflow region of the shunt is in fluid communication with an anterior chamber of the eye and the outflow region is in fluid communication with a drainage location; and
an implantable sensor configured to measure an intraocular pressure of the eye,
wherein—
the flow control element is in a first position when the adjustable flow shunt is implanted, and
the flow control element is configured to transition between the first position and a second, different position that enables increased fluid flow between the anterior chamber and the drainage location relative to the first position (a) when the measured intraocular pressure exceeds a predetermined threshold, (b) after a predetermined time-period following implantation has elapsed, or both (a) and (b).

2. The system of claim 1 wherein the flow control element is configured to transition to the second position when the measured intraocular pressure exceeds the predetermined threshold.

3. The system of claim 2 wherein the predetermined threshold is between about 18 mmHg and about 28 mmHg.

4. The system of claim 1 wherein the flow control element is configured to transition to the second position after the predetermined time-period following implantation has elapsed.

5. The system of claim 4 wherein the predetermined time-period is between about one week and about eight weeks.

6. The system of claim 4 wherein the predetermined time-period is between about four weeks and about six weeks.

7. The system of claim 1 wherein the flow control element is configured to transition to the second position only once both (a) the measured intraocular pressure exceeds the predetermined threshold and (b) the predetermined time-period following implantation has elapsed.

8. The system of claim 1 wherein the flow control element is configured to automatically transition to the second position.

9. A system for treating glaucoma, the system comprising:
an adjustable flow shunt having an inflow region, an outflow region, and a flow control element configured to control fluid flow through the shunt, wherein, when implanted into an eye—
the inflow region is in fluid communication with an anterior chamber of the eye, the outflow end region is in fluid communication with a drainage location, and the device directs the flow of aqueous from the anterior chamber to the drainage location, and
the flow control element is transitionable between at least a first position enabling a first amount of aqueous to flow through the shunt and a second position enabling a second amount of aqueous different than the first amount to flow through the shunt; and
an implantable pressure sensor configured to intermittently measure a pressure value indicative of an intraocular pressure at a predetermined time interval;
wherein the system is configured such that—
if the determined pressure value exceeds a first predetermined threshold, (i) the system moves the flow control element toward the second position, or (ii) the system generates a notification instructing a user to move the flow control element toward the second position, and
if the determined pressure value falls below a second predetermined threshold, (iii) the system moves the flow control element toward the first position, or (iv) the system generates a notification instructing a user to move the flow control element toward the first position.

10. The system of claim 9 wherein the flow control element has a plurality of discrete positions between the first position and the second position, and wherein each of the plurality of discrete positions enables a different amount of aqueous to flow through the shunt.

11. The system of claim 9 wherein the predetermined time interval is daily.

12. The system of claim 9 wherein the predetermined time interval is weekly.

13. The system of claim 9 wherein the first predetermined threshold is between about 18 mmHg and about 28 mmHg.

14. The system of claim 9 wherein the second predetermined threshold is between about 5 mmHg and about 12 mmHg.

15. The system of claim 9 wherein the sensor is physically coupled to the shunt.

16. The system of claim 9 wherein the sensor is wirelessly coupled to the shunt.

17. The system of claim 9 wherein if the determined pressure value exceeds the first predetermined threshold, the system automatically moves the flow control element toward the second position, and wherein if the determined pressure value falls below the second predetermined threshold, the system automatically moves the flow control element toward the first position.

18. A computer-implemented method of altering fluid flow through an adjustable flow shunt in the treatment of glaucoma, the shunt having a flow control element controlling fluid flow through the shunt, the computer-implanted method comprising:
directing a pressure sensor implanted in an eye to measure a pressure value indicative of an intraocular pressure;
receiving, from the pressure sensor, the pressure value indicative of the intraocular pressure; and
determining, based at least in part on the received pressure value, whether to adjust a position of the flow control element to adjust the fluid flow through the shunt.

19. The computer-implemented method of claim 18, further comprising displaying, via a display element, the pressure value.

20. The computer-implemented method of claim 18 wherein determining whether to adjust a position of the flow control element comprises automatically determining whether to adjust a position of the flow control element based on one or more criteria.

21. The computer-implemented method of claim 20 wherein the one or more criteria includes a pressure range.

22. The computer-implemented method of claim 20, further comprising automatically directing an actuator to adjust the position of the flow control element based on a determination to adjust the flow control element.

23. The computer-implemented method of claim 20, further comprising instructing a user to adjust the flow control element based on a determination to adjust the flow control element.

24. A system for draining fluid from a first body region to a second body region, the system comprising:
an adjustable flow shunt having an inflow region, an outflow region, and a flow control element configured to control fluid flow through the shunt, wherein, when implanted into the patient—
the inflow region is in fluid communication with the first body region, the outflow end region is in fluid communication with the second body region, and the device directs the flow of fluid from the first body region to the second body region, and
the flow control element is transitionable between at least a first position enabling a first amount of fluid to flow through the shunt and a second position enabling a second amount of fluid different than the first amount to flow through the shunt; and
an implantable pressure sensor configured to intermittently measure a pressure value indicative of a pressure in the first body region at a predetermined time interval;
wherein the system is configured such that—
if the determined pressure value exceeds a first predetermined threshold, (i) the system moves the flow control element toward the second position, or (ii) the system generates a notification instructing a user to move the flow control element toward the second position, and
if the determined pressure value falls below a second predetermined threshold, (iii) the system moves the flow control element toward the first position, or (iv) the system generates a notification instructing a user to move the flow control element toward the first position.

25. The system of claim 24 wherein the flow control element has a plurality of discrete positions between the first position and the second position, and wherein each of the plurality of discrete positions enables a different amount of fluid to flow through the shunt.

26. The system of claim 24 wherein the predetermined time interval is daily.

27. The system of claim 24 wherein the predetermined time interval is weekly.

28. The system of claim 24 wherein the first predetermined threshold is between about 18 mmHg and about 28 mmHg.

29. The system of claim 24 wherein the second predetermined threshold is between about 5 mmHg and about 12 mmHg.

30. The system of claim 24 wherein the sensor is physically coupled to the shunt.

31. The system of claim 24 wherein the sensor is wirelessly coupled to the shunt.

32. The system of claim 24 wherein if the determined pressure value exceeds the first predetermined threshold, the system automatically moves the flow control element toward the second position, and wherein if the determined pressure value falls below the second predetermined threshold, the system automatically moves the flow control element toward the first position.

33. A system for draining fluid from a first body region to a second body region, the system comprising:
an adjustable flow shunt having an inflow region, an outflow region, and a flow control element configured to control the flow of fluid through the adjustable flow shunt, wherein the adjustable flow shunt is configured to be implanted into a patient such that the inflow region of the shunt is in fluid communication with the first body region and the outflow region is in fluid communication with the second body region; and
an implantable sensor configured to measure a pressure in the first body region,
wherein—
the flow control element is in a first position when the adjustable flow shunt is implanted, and
the flow control element is configured to transition from the first position to a second, different position that enables increased fluid flow between the first body region and the second body region relative to the first position (a) when the measured pressure exceeds a predetermined threshold, (b) after a predetermined time-period following implantation has elapsed, or both (a) and (b).

34. The system of claim 33 wherein the flow control element is configured to transition to the second position when the measured pressure exceeds the predetermined threshold.

35. The system of claim 34 wherein the predetermined threshold is between about 18 mmHg and about 28 mmHg.

36. The system of claim 33 wherein the flow control element is configured to transition to the second position after the predetermined time-period following implantation has elapsed.

37. The system of claim 36 wherein the predetermined time-period is between about one week and about 8 weeks.

38. The system of claim 36 wherein the predetermined time-period is between about four weeks and about six weeks.

39. The system of claim 33 wherein the flow control element is configured to transition to the second position only once both (a) the measured pressure exceeds the predetermined threshold and (b) the predetermined time-period following implantation has elapsed.

40. The system of claim 33 wherein the flow control element is configured to automatically transition to the second position.
